(19) United States
(12) Patent Application Publication
(10) Pub. No.: US 2018/0110560 A1
(43) Pub. Date: Apr. 26, 2018

(54) MINIMALLY INVASIVE SURGICAL DEVICES

(71) Applicant: ZAMENIS SURGICAL LLC, Newark, DE (US)

(72) Inventors: JOSEPH BENNETT, Newark, DE (US); Quirk Ryan, Newark, DE (US); Lipman Ethan, West Babylon, NY (US); Penman Jessica, Guilford, CT (US); Freebery Mallory, Hockessin, DE (US)

(73) Assignee: ZAMENIS SURGICAL LLC, Newark, DE (US)

(21) Appl. No.: 15/565,918

(22) PCT Filed: May 10, 2016
(86) PCT No.: PCT/US16/31604

§ 371 Date: Oct. 12, 2017

Related U.S. Application Data
(60) Provisional application No. 62/159,622, filed on May 11, 2015.

(51) Int. Cl. A61B 18/14

(52) U.S. Cl. CPC A61B 18/1445; A61B 2017/2929; A61B 2018/1455; A61B 18/1482

(57) ABSTRACT

A minimally invasive surgical instrument includes a housing and an outermost elongated shaft having an end effector operatively coupled to a distal end thereof. The outermost elongated shaft is releasably secured to a pincher within the housing of the instrument such that during the course of a surgical procedure, an appropriately dimensioned elongated shaft and suitable end effector may be interchanged as desired. The elongated shaft may have an adjustable length, and the end effectors of the elongated shaft may be interchangeable.

### CROSS REFERENCE TO RELATED APPLICATIONS

This application relates to and claims priority from U.S. Provisional Patent Application Ser. No. 62/159,622 filed on May 11, 2015, the entire contents of which are hereby incorporated by reference.

### FIGURE SELECTED TO PUBLICATION

FIG. 4

### BACKGROUND TO THE INVENTION

### Technical Field

The present disclosure generally relates to surgical instruments for performing a minimally invasive surgical procedure (e.g., a laparoscopic or an endoscopic procedure), and more particularly to a minimally invasive surgical instrument that includes interchangeable shafts and/or end effectors.

### Description of the Related Art

Minimally invasive surgery has increasingly become the norm for surgeries that were previously performed through open surgical techniques. During a minimally invasive surgical procedure, access to the surgical site is provided via one or more openings of a small diameter that are made within the body wall. Since relatively small openings are made during a minimally invasive surgery as compared to an open surgery, minimally invasive surgery is advantageous over traditional open surgical procedures because they typically offer a taster recovery time and a better cosmetic result.

Minimally invasive surgical tools typically include an elongated shaft including a distal end at which an end effector (e.g., jaw members) is coupled to the elongated shaft. An appropriately sized tool including the end effector and elongated shaft should be selected by taking into consideration a person's body size, the location of the tissues within the person's body, and the specific procedure to be performed.

An example of a prior art minimally invasive surgical tool is described with reference to FIG. 1 which shows a minimally invasive surgical tool A that includes an elongated shaft S and en end effector E (e.g., a blade) that is clamping and cutting tissue T. While the elongated shaft and end effectors may come in different sizes (e.g., lengths), the particular elongated shaft and end effector cannot be changed mid-surgery. That is, the shafts and/or end effectors are not replaceable and/or interchangeable. Thus, oftentimes a surgeon must have multiple devices and tools of various sizes and lengths available during the procedure since any one device or tool may not be readily adjusted mid-surgery. Having the right tool at the surgeon's disposal when needed would shorten the total time of operation.

Typically, during a minimally invasive surgery, specialized tools are needed that facilitate grasping and clamping tissue, cauterizing tissue to coagulate blood, and cutting tissue. Such instruments may include, for example, electrosurgical forceps, which utilize both mechanical clamping action and electrical energy to effect hemostasis by heating, tissue and blood vessels to coagulate, cauterize, and/or seal tissue. As shown in FIG. 1, a demonstration of tissue sealing is shown in which a minimally invasive surgical instrument or endoscopic instrument A includes an elongated shaft S includes an end effector E at a distal end thereof. The end effector E may include a pair of jaws and a blade such that tissue T that is clamped between the jaws of the end effector E may be severed by the blade of the end effector E and may then be cauterized by the end effector.

There is a continuing need for a minimally invasive surgical device that is multifunctional, adaptable, and/or modifiable to meet changing needs during the course of a surgical procedure such that the necessity of introducing other surgical devices mid-surgery is minimized. It is to be understood that nothing described in the background section is to be construed as an, admission of prior art unless explicitly stated otherwise.

### ASPECTS AND SUMMARY OF THE INVENTION

In an embodiment, a minimally invasive surgical instrument may include a housing having a opening. The interior of the housing may be accessed via the opening, and a pincher configured to secure an elongated shaft to the housing may be disposed on an interior surface of the housing at or near the opening of the housing. The elongated shaft may include an outermost shaft and an end effector shaft, and may have an overall length that is adjustable (e.g., by telescoping motion of the outermost shaft and the end effector shaft relative to one another).

A pincher including a first arm member and a second arm member that are pivotable relative to one another such that the pincher is transitionable between an open position and a closed position to grip the outermost elongated shaft extending through the opening of the housing. The outermost elongated shaft may include a first collar and a second collar that are spaced apart from one another along a length of the outermost elongated shaft. The first and second arms of the pincher may be secured around a circumference of the outermost elongated shaft and may engage the collars of the outermost elongated shaft to prevent axial movement of the outermost elongated shaft relative to the housing when the pincher is in the closed position. An end effector shaft may be slidable through the outermost elongated shaft, and an end effector may be operatively coupled to the end effector shaft, the end effector being actuated by translation of the end effector shaft relative to the outermost elongated shaft. A knife blade shaft being translatable through the end effector shaft, the knife blade being operatively coupled to a distal end of the knife blade shaft and being translatable though the end effector in response to a distal translation of the knife blade shaft. The end effector includes an electrically conductive surface configured to cauterize tissue coming into contact with the end effector, and the end effector is releasably secured to the end effector shaft.

A minimally invasive surgical instrument may include a housing that may include an opening and a pincher that is disposed within the housing. The pincher may include a first arm member and a second arm member that are pivotable with respect to one another to transition between an open position and a closed position; and an outer elongated shaft that includes a first collar and a second collar. The pincher may be configured to clamp onto the outer elongated shaft and to secure the outer elongated shaft to the housing. The pincher may be sized and/or dimensioned to be securely received between the first collar and the second collar such that when the pincher is in the closed position, the first and second collars prevent relative of the outer elongated shaft and the pincher along a longitudinal axis extending through the outer elongated shaft.

An end effector shaft may be translatable through the outer elongated shaft, and a cutting blade shaft may be translatable through the end effector shaft. An end effector may be releasably coupled to the end effector shaft, and the end effector may be releasable from the end effector shaft while the end effector shaft remains secured to the housing. A locking mechanism may releasably secure a connection member of the end effector to the end effector shaft. The locking mechanism may comprise a latch and a hook and/or a camming member. The connection member may be rotated in a first direction relative to the end effector shaft such that the camming member locks the connection member to the end effector shaft and when the connection member is rotated in a second direction relative to the end effector shaft, the camming member may unlock the connection member from the end effector shaft. The locking mechanism may lock the end effector to the end effector shaft when by being rotated in a first direction relative to the other and is unlocked by being rotated in a second direction. The connection member may be threadably secured to the end effector shaft. The connection member may be frictionally secured within the end effector shaft. The end effector may include an electrically conductive surface configured to cauterize tissue coming into contact with the end effector. The connection member may include an L-shaped groove for receiving and releasably locking a protrusion extending from the end effector.

The end effector shaft may be configured to be coupled to an actuator within the housing. Translation of the actuator may actuate the end effector, and the end effector shaft may include a collar that includes a plurality of fingers/recesses and the actuator may include a plurality of corresponding fingers/recesses that may be configured to mate with one another. The end effector shaft may include at least two shafts that are telescopically arranged such that a length of the end effector shall is adjustable. A locking mechanism may secure the two shafts in a relative position with respect to one another. The locking mechanism may include outwardly biased buttons that are provided on the inner shaft and receptacles that are provided on the outer shaft, the receptacles being configured to receive the outwardly biased buttons. The at least two shafts may include an outer shaft and an inner shaft. The outer shaft may include a track and the inner shaft may include a protrusion that is able to travel along the track when the two shafts are positioned in a first rotational position relative to one another such that the two shafts may slide relative to one another and are inhibited form sliding relative to one another when the two shafts are in a second rotational position relative to one another. The locking mechanism may include a hook and a latch.

The above and other aspects, features and advantages of the present disclosure will become apparent from the following description read in conjunction with the accompanying drawings, in which like reference numerals designate the same elements.

### BRIEF DESCRIPTION OF THE DRAWINGS

FIG. 1 is a perspective view of a prior art minimally invasive surgical device being used to cut tissue;

FIG. 2 is a front view of a minimally invasive surgical instrument in accordance with the present disclosure;

FIG. 3A is a perspective view of an outermost shaft in accordance with the present disclosure;

FIG. 3B is a perspective view of an end effector shaft in accordance with the present disclosure;

FIG. 3C is a perspective view of a cutting blade shaft in accordance with the present disclosure;

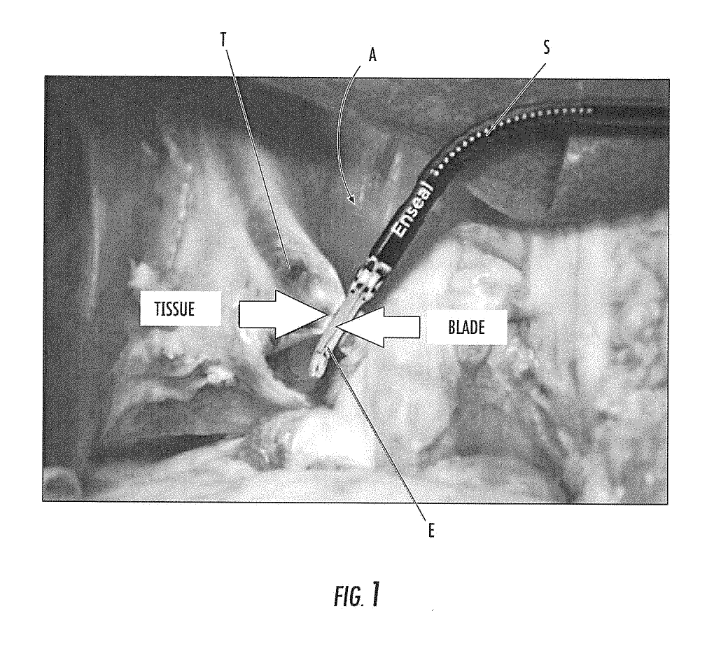

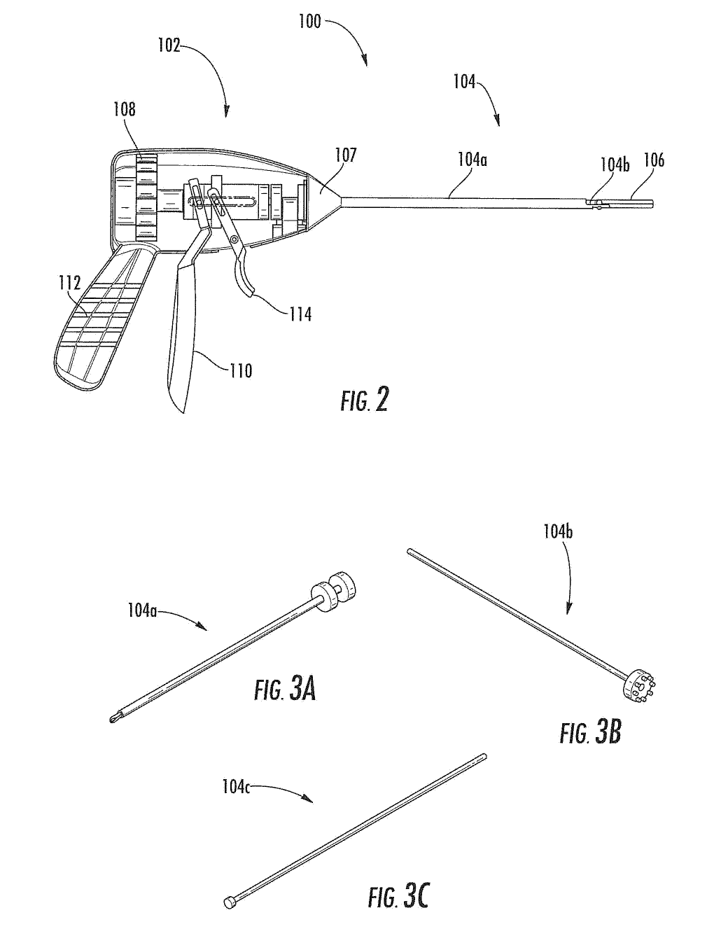

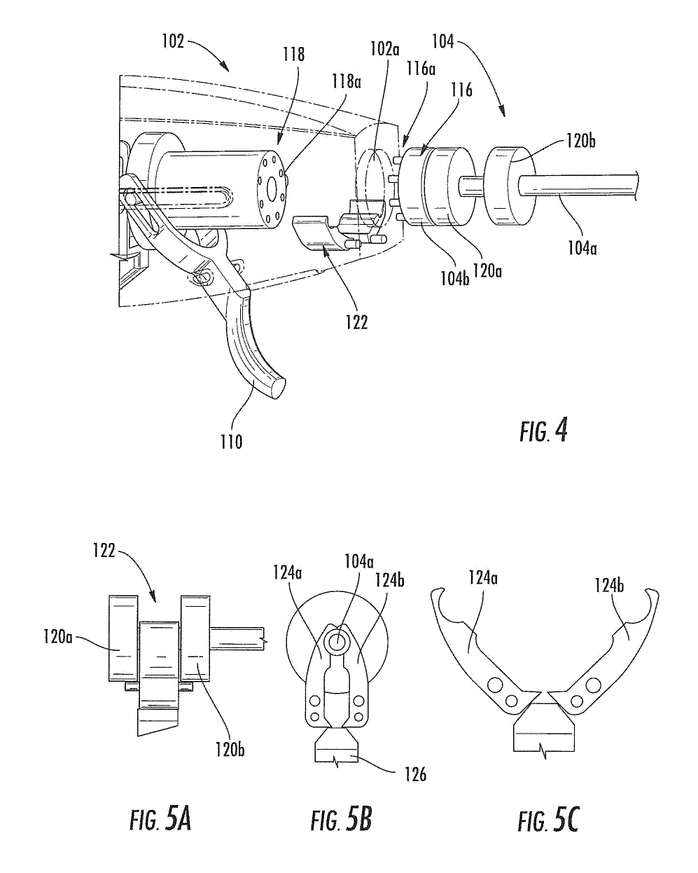

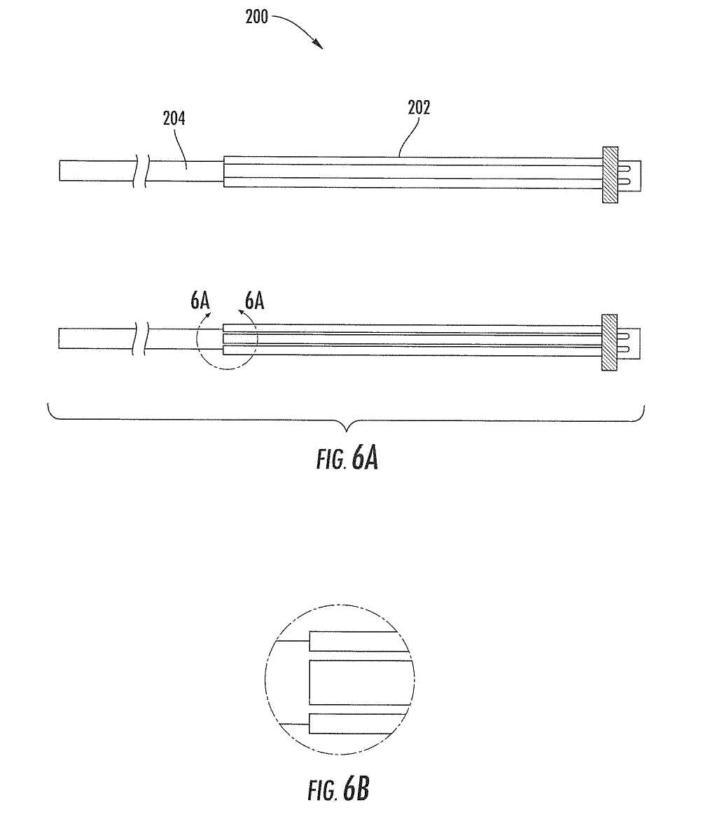

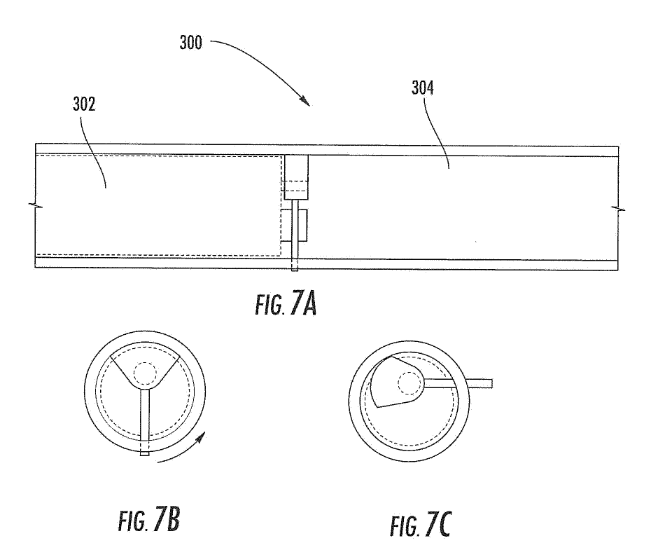

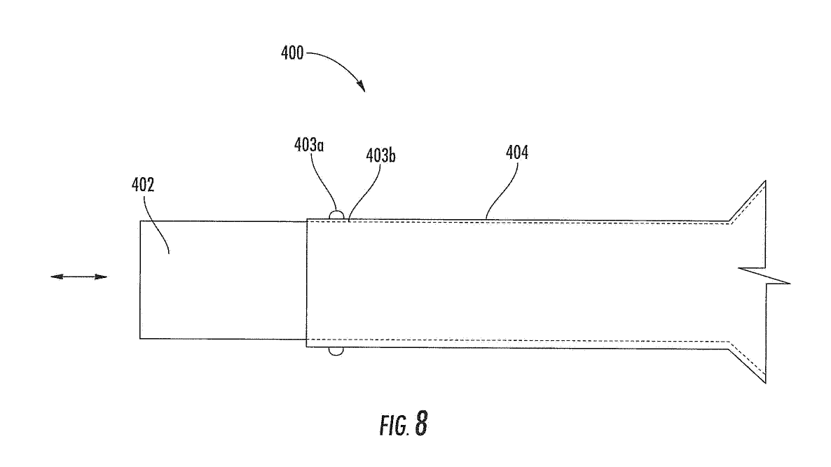

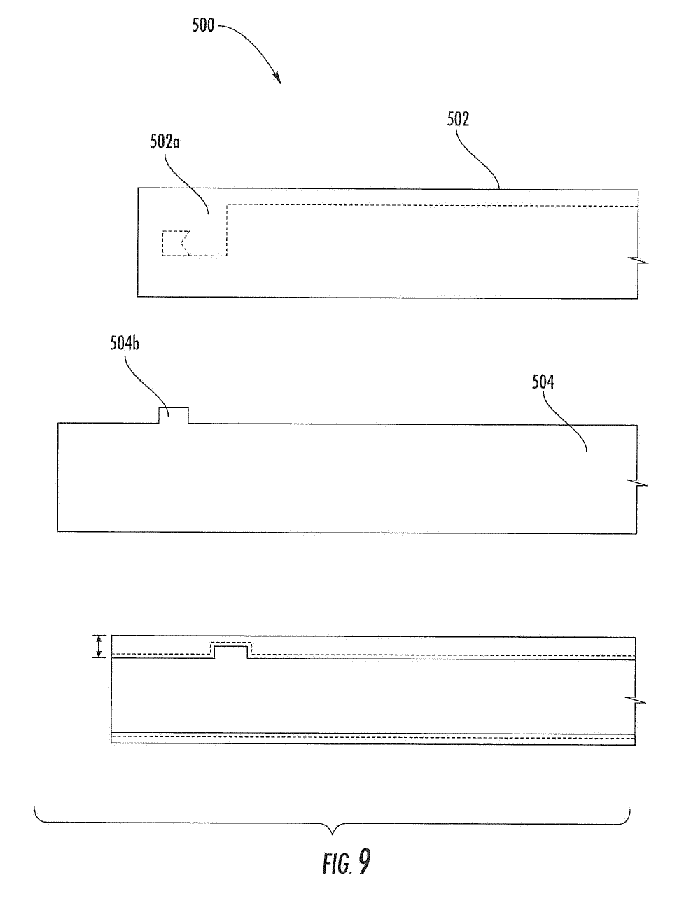

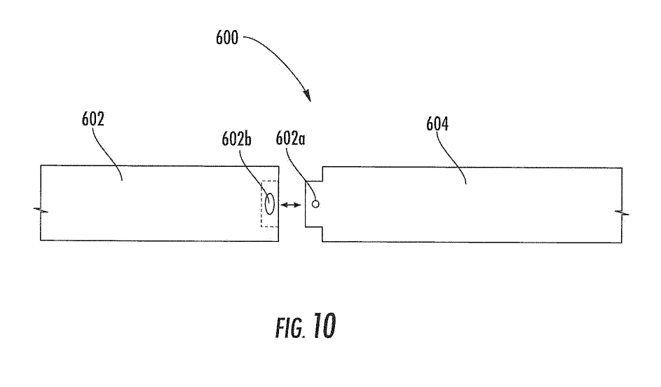

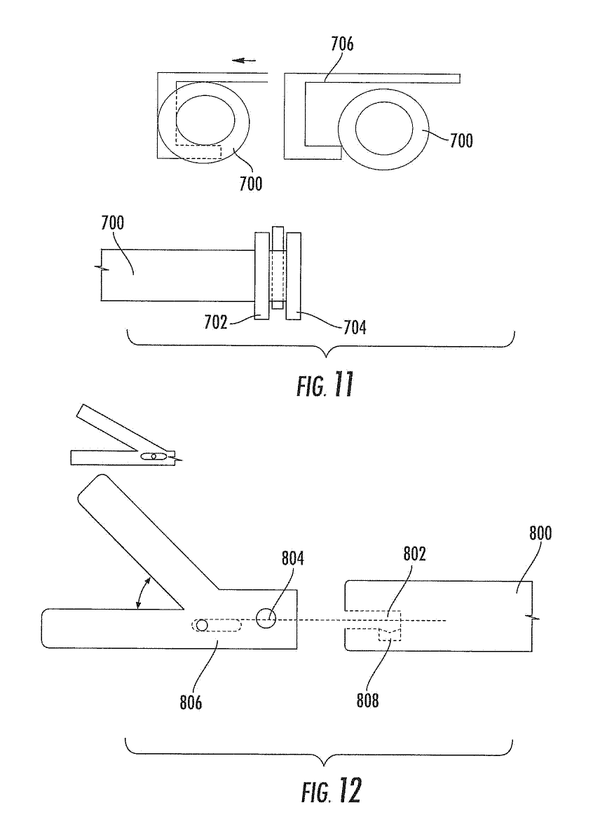

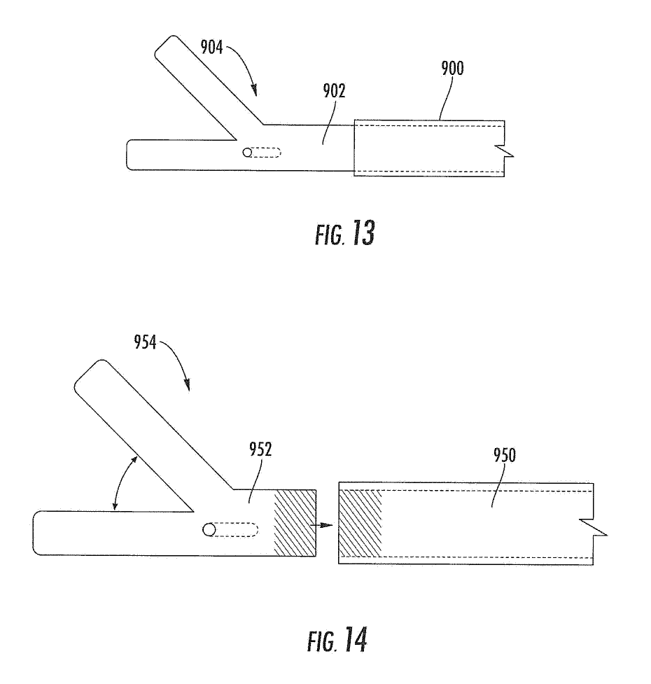

FIG. 4 is a partial exploded view of the minimally invasive surgical instrument of FIG. 2 with some parts exploded outwardly for illustrative purposes;

FIGS. 5A-5C are view of a pincher shown in various states of engagement with the push button engagement noting the driving engagement motion process;

FIG. 6 includes various views of an elongated shaft in accordance with the present disclosure;

FIGS. 7A-7C are various views of an elongated shaft in accordance with the present disclosure;

FIG. 8 is a front view of an elongated shaft having an adjustable length in accordance with the present disclosure;

FIG. 9 are various views of an elongated shaft having an adjustable length in accordance with the present disclosure;

FIG. 10 is a front view of a locking mechanism to secure an elongated shaft to an end effector in accordance with the present disclosure;

FIG. 11 is a front view of a locking mechanism to secure an elongated shaft to an end effector in accordance with the present disclosure;

FIG. 12 is a front view of a locking mechanism to secure an elongated shaft to an end effector in accordance with the present disclosure;

FIG. 13 is a front view of a locking mechanism, to secure an elongated shaft to an end effector in accordance with the present disclosure; and

FIG. 14 is a front view of a locking mechanism to secure an elongated shaft to an end effector in accordance with the present disclosure.

### DETAILED DESCRIPTION

Exemplary embodiments of the present disclosure are described below with reference to the accompanying drawings. Various exemplary embodiments of the present disclosure are illustrated in the accompanying drawings and a related detailed description is stated. It should be understood that various modifications may be made to the exemplary embodiments without departing from the scope and spirit of the present disclosure. Accordingly, it should be understood that the various exemplary embodiments of the present disclosure are not intended to limit a specific embodiment form, and include all possible modifications or equivalents or substitutes within the spirit and. technological scope of the present disclosure. In relation to a description of the drawing, like reference symbols denote like constituent elements.

The terms and words used in the following description and claims are not limited to the bibliographical meanings but are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

As used herein, the singular forms “a”, “an”, and “the” are intended to include the plural forms, including “at least one”, unless the content clearly indicates otherwise. “Or” means “and/or”. As used herein, the term “and/or” includes any and all combinations of one or more of the associated listed items. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. As used herein, the term “distal” refers to the portion that is being described which is further from a user while the term “proximal” refers to the portion that is being described which is closer to a user.

Referring to FIG. 2, a minimally invasive surgical device 100 will now be described. The device 100 may include a housing 102 and an elongated shaft 104 having an end effector 106, e.g., sealing forceps, at a distal end thereof. Advantageously, the elongated shaft 104 and the end effector 106 coupled thereto may be replaced during the surgical procedure thus eliminating the need to introduce another surgical instrument in situations where a variety of dimensioned (e.g., lengths, etc.) of elongated shafts are required during the course of the surgical procedure. For example, pressing of button 126 releases members and thus releases elongated shaft 104, as will be discussed below in farther detail.

The elongated shaft 104 may include an outermost shaft 104a (FIG. 3A), an end effector shaft 104b (FIG. 3B) slidably disposed within the outermost shaft 104, and a cutting blade shaft 104c (FIG. 3C) slidably disposed within the end effector shaft 104b. An end effector 106 (e g., sealing forceps) may be permanently or releasably coupled to a distal end of the end effector shaft 104b. The elongated shaft 104 may be releasably coupled, via an engagement system to be discussed in detail, to the housing 102 such that during the course of a surgical procedure, the elongated shaft 104 may be substituted for another elongated shaft 104 that is similar but differently dimensioned (e.g., length, diameter, etc.) or that may have a different end effector 106 depending upon the desired function or procedure to be performed. The elongated shaft 104 may be formed from a rigid material and may be able to withstand forces (e.g., 5 lbs of pressure) that may be applied to its distal end without bending. Alternatively, the elongated shaft 104 may be capable of being bent into a desired shape or contour such as the curved shape of the elongated shaft S shown in FIG. 1). The elongated shaft 104 may have an overall length within a range of 20 cm and 40 cm inclusive. A removable cover or cap 107 may be placed at a distal end of the housing 102 and may facilitate maintaining the elongated shaft 104 in a coupled configuration with the housing 102.

Upon engagement, the elongated shaft 104 may be operatively coupled to an exposed rotating wheel 108 such that rotation of the rotating wheel 108 in a clockwise or in a counterclockwise direction may effect a corresponding rotation of the elongated shaft 104, and related components, relative to a longitudinal axis of the elongated shaft 104. As a result, a precise finger-touch-control allows for minute rotational adjustment control, and thus additionally control of the implement at the end of elongated shaft 104.

The end effector 106 may include a pair of jaw members 106a, 106b that are pivotably connected to one another. Each of the jaw members 106a, 106b may include a channel 107 through which a knife blade (not shown) may travel such that when tissue is clamped between the jaw members 106a, 106b, the knife blade may translate through the channel 107 to sever or cut the clamped tissue. The jaw members 106a, 106b may be biased toward an open state such that in a relaxed state the jaw members 106a, 106b define an angle (e.g., 45 degrees) relative to the longitudinal axes of the jaw members 106a, 106b. The jaw member 106b may remain stationary while the jaw member 106a pivots with respect to the jaw member 106b. Pivotal movement of the jaw member 106b may be controlled by the outermost shaft 104a as relative movement of the outermost shaft 104a and the end effector shaft 104b may effect pivotal movement of the jaw members 106a, 106b relative to one another.

Movement of a jaw trigger 110 relative to a handle 112 of the housing 102 may cause the relative pivotal movement of the jaw members 106a, 106b. The jaw trigger 110 may be operatively coupled to the outermost shaft 104a, which may be pivotably connected to the jaw member 106a at point A. In particular, squeezing the jaw trigger 110 in a direction toward a handle 112 of the housing 102 may cause the outermost shaft 104a to retract through the housing 102 thereby thereby pulling on the jaw member 106a at point A and causing it to pivot at point B where it is pivotably connect to jaw member 106b. Accordingly, movement of the jaw trigger 110 toward handle 112 pivots the jaw members 106a, 106b toward one another, whereas movement of the jaw trigger 110 back to its initial position apart from the handle 112 causes the jaw members 106a, 106b to return to the open position. The jaw members 106a, 106b may be able to apply a force (e.g., 50 lbs of pressure) to tissue clamped therebetween.

When the jaw members 106a, 106b are in the clamped position, pivoting a blade trigger 114 in a direction toward the handle 112 may cause a knife blade (not shown) disposed at a distal end of the cutting blade shaft 104c to travel through the shaft 104 and through the channels 107 of the jaw members 106a, 106b. The blade trigger 114 may be biased in a direction away from the handle 112. Electrical wiring (not shown) may be disposed within the shaft 104 and may be electrically connected to the end effector 106 such that electrical energy may be passed to the jaw members 106a, 106b to cauterize any desired tissues. In particular, the housing 102 may contain a bipolar electrode and wiring that are operatively coupled to the end effector 106. A switch (not shown) may be selectively actuated to provide electrical power to the bipolar electrode.

Referring to FIG. 4, when the cap 107 is removed from the housing 102, the elongated shaft 104 may be removed or coupled to the housing 102 via a portal or opening 102a of the housing, which provides access to the interior of the housing 102. The end effector shaft 104b may include a collar 116 at its proximal end that is configured to be coupled to an actuator (or jaw closer) 118 is longitudinally translatable within the housing upon actuation of the trigger 110, such that actuation of the trigger 110 causes the actuator 118 to translate distally, which in turn causes the end effector shaft 104b to move distally and results in actuation of the end effector 106. The collar 116 and the actuator 118 may include complementary mating parts to ensure a secure and releasable relationship between them. For example, the collar 116 of the end effector shaft 104b may include a plurality of fingers 116a (e.g., 8 fingers) that are configured to mate with recesses 118a of the actuator 118. The outermost shaft 104a may include two collars 120a, 120b that have a space therebetween.

A pincher system 122 may be disposed at or near the distal end of the housing 102 proximal to the opening 102a may be configured to releasably secure the portion of the outermost shaft 104a between the collars 120a, 120b. In particular, the pincher system 122 may include arms 124a, 124b that are pivotable relative to one another. Each of the arms 124a, 124b may be pivotally connected to an inner surface of the housing 102 near the opening 102a. The pincher or pincher system 122 may be biased, for example by a spring or biasing element engaging each arm, toward a closed state in which the arms 124a, 124b are in a closed relationship relative to one another (FIG. 5B, see arrows and with button 126 in a recessed position). To counter the urging biasing element that urges closure between arms 124a, 124b, a button 126 is provided and available externally (FIG. 4) and extends through the housing and is operatively coupled to or in contact with proximal ends of the arms 124a, 124b such that when the button 126 is pressed upwardly (FIG. 5C) in a direction Z toward the interior of the housing 102, the button 126 interacts with proximal surfaces of the arms 124a, 124b and causes the arms 124a, 124b to pivot toward an open position (moving from FIG. 5B to FIG. 5C). The proximal ends of the arms 124, 124b may have a curvature and the distal end of the button 126 may be tapered such that when the button 126 is moved inwardly (in direction Z toward the housing 102), the arms 124a, 124b are urged to pivot toward an open position, as shown in FIG. 5C. Therefore, it will be understood that to remove shaft 104 and related elements, the user can press button 126 upwardly (FIG. 5C) relative to the housing, thus urging an opening of arms 124a, 124b and moving them from a closed position in FIG. 5B to the open position in FIG. 5C, allowing a user to grasp and remove the same.

The arms 124a, 124b may have any suitable shape (e.g., curvature) that corresponds to the shape of the shaft 104a such that the arms 124a, 124 may pinch the shaft 104a around the circumference thereof when the arms are in the closed position such that they apply a sufficient force to maintain the elongated shaft within the housing 102. Moreover, when the arms 124a, 124b are closed, the collars 120a, 120b interact with the arms 124a, 124b which may be sired to correspond with the space between the collars 120a, 120b such that movement of translation of the shaft 104a is impeded or prevented in a longitudinal direction relative to the housing 102.

In other embodiments, elongated shafts that may be substituted for the elongated shaft 104 will now be described in which the elongated shafts are substantially the same as the shaft 104 except that they are also configured to have an adjustable length without necessitating removal of the shaft from the housing 102 once coupled thereto, in particular, elongated shafts 200-500 described with reference to FIGS. 6-9 have an overall length that is adjustable via a variety of mechanisms including, for example, a telescoping mechanism including an outer shaft and an inner shaft that is slidably and controllably received within the outer shaft, along a lengthwise direction thereof, such that the overall length may be adjusted as desired.

As shown in FIG. 6, an elongated shaft 200 may include an outer shaft 202 and an inner shaft 204 that are configured such that the shafts 202, 204 may move in a telescoping motion relative to one another. For example, an end effector shaft (such as those described herein) may be comprised of the shafts 202, 204 such that an end effector coupled thereto may be distally translated to a desired distance.

As shown in FIG. 7, an elongated shaft 300 may include an inner shaft 302 and an outer shaft 304 that are slidably coupled to one another. Each of the shafts 302, 304 includes cammed surfaces that are configured to lock relative translation of the shafts 302, 304 when the shafts are rotated in a first direction relative to one another (FIG. 7B) and are free to slide relative to one another when rotated in a second direction relative to one another (FIG. 7C).

As shown in FIG. 8, yet another elongated shaft 400 includes an inner shaft 402 and an outer shaft 404 that are slidably coupled to one another, for example in a telescopic arrangement. The inner and outer shafts 402 and 404 may be releasably locked relative to one another via a locking mechanism. For example, the inner shaft 402 may include spring loaded or outwardly biased buttons 403a that are configured to interact with receptacles 403b disposed on the outer shaft 404 such that when the buttons 403a engage the receptacles 403b sliding of the shafts 402, 404 relative to one another is prevented, and when the buttons 403a are withdrawn from the receptacles 404, the shafts 402, 404 are free to slide relative to one another.

As shown in FIG. 9, an elongated shaft 500 may include an outer shaft 502 and an inner shaft 504 that are slidably coupled to one another. The outer shaft 502 may include a track 502a through which an extrusion or protrusion 504b may travel. When the shafts 502, 504 are positioned in a first position relative to one another, the protrusion 504b is prevented from traveling through the track 502a, but when the shafts 502, 504 are rotated relative to one another the protrusion 504b may freely move through the track 502a and the shafts 502, 504 may slide relative to one another.

Various locking mechanisms for having an interchangeable end effector that is releasably secured to an elongated shaft are shown and described with reference to FIGS. 10-14 in which end effectors are shown having an releasable connection point to an end of a elongated shaft, similar in other respects to the elongated shaft 104 discussed above except in the respects specifically discussed.

In particular, as shown in FIG. 10, an elongated shaft 604 may include a locking button 602a that is configured to be releasably secured within a receptacle 602b of a connection member 602 of an end effector. Preferably, the locking button 602 is biased toward a locked position and may be transitioned to an unlocked position via a button (not shown) on the handle 112 of the housing 102 or in another accessible location of the housing 102.

In another embodiment, as shown in FIG. 11, an elongated shaft 700 (similar to the elongated shaft 104 discussed above) may include a locking member that includes two parallel spaced apart collars 702, 704 that are configured to receive snuggly therebetween a latch 706 of an end effector (not shown) to releasably secure the end effector to the elongated shaft 700. Although described as securing an end effector to an elongated shaft, the locking mechanism illustrated in FIG. 11 may alternatively be used to releasably secure an elongated shaft thereto. For example, an elongated shaft may include at least two sections that may be secured one to the other via the hook latch mechanism illustrated in FIG. 11. For example, a latch may include a groove that is provided on one shaft and a hook may be provided on another shaft such that the two shafts may be coupled together.

Alternatively, in another embodiment shown in FIG. 12, an elongated shaft 800 (similar in other respects to shaft 104) may include a track 802 that is configured to releasably receive a securing protrusion 804 of an end effector 806 within a groove 808 of the of the track 802, wherein the protrusion 804 is freed from the groove 808 via a twisting motion in which the end effector 806 and the elongated shaft 808 are rotated relative to one another with respect to their longitudinal axes. Inadvertent disconnection of the end effector 806 from the elongated shaft 808 may be minimized via frictional forces between the protrusion 804 of the end effector 806 and the groove 808, which snuggly receives the protrusion 804 therein.

Alternatively, in another embodiment, an elongated shaft 900 (similar in other respects to the shaft 104) may include a receiving channel extending along its interior along a length thereof to receive a somewhat smaller diameter shaft 902 extending from a proximal end of an end effector 904 such that the shaft 902 forms a tight connection with the elongated shaft 900 and cannot be removed from the shaft 900 without application of a sufficient force to effect such separation. Such a connection may be substantially permanent or may be releasable.

Alternatively in yet another embodiment, an elongated shaft 950 (similar in other respects to the shaft 104) may include a receiving channel extending along its interior along a length thereof to receive a somewhat smaller diameter shaft 952 extending from a proximal end of an end effector 954 such that the shaft 952 forms a threaded connection with the elongated shaft 950 in which the shaft 952 has an external threading on a proximal end thereof and the shaft 950 has an internal threading on a distal end thereof in which the shafts 950, 952 that are configured to engage or disengage with one another via a twisting motion is one of a clockwise or counterclockwise direction relative to one another.

Although exemplary embodiments of the present disclosure have been described in detail hereinabove, it should be clearly understood that many variations and modifications of the basic inventive concepts herein described, which may appear to those skilled in the art, will still fall within the spirit and scope of the exemplary embodiments of the present invention as defined in the appended claims.

What is claimed is:

1. A minimally invasive surgical instrument, comprising:
a housing, the housing including an opening;
a pincher being disposed within the housing, the pincher comprising a first arm member and a second arm member that are pivotable with respect to one another to transition between an open position and a closed position;
an elongated shaft comprising an outermost elongated shaft and an end effector shaft, the outermost elongated shaft being releasably coupled to the housing and extending through the opening of the housing, the first and second arm members releasably securing the outermost elongated shaft to the housing, the outermost elongated shaft including a first collar and a second collar spaced apart from one another along a length of the outermost elongated shaft, the first and second arms of the pincher being positioned between the first and second collars to prevent axial movement of the outermost elongated shaft relative to the housing when the pincher is in the closed position, the end effector shaft being slidable through the outermost elongated shaft;
an end effector being operatively coupled to the end effector shaft, the end effector being actuated by translation of the end effector shaft relative to the outermost elongated shaft; and
a knife blade shaft being translatable through the end effector shaft, the knife blade being operatively coupled to a distal end of the knife blade shaft and being translatable though the end effector in response to a distal translation of the knife blade shaft,
wherein the elongated shaft has an overall length that is adjustable;
wherein the end effector includes an electrically conductive surface configured to cauterize tissue coming into contact with the end effector; and
wherein the end effector is releasably secured to the end effector shaft

2. A minimally invasive surgical instrument, comprising:
a housing, the housing including an opening;
a pincher being disposed within the housing, the pincher comprising a first arm member and a second arm member that are pivotable with respect to one another to transition between an open position and a closed position; and
an outer elongated shaft including a first collar and a second collar, the outer elongated shaft having a longitudinal axis extending therethrough,
wherein the pincher is configured to clamp onto the outer elongated shaft and to secure the outer elongated shaft to the housing, and wherein the pincher is dimensioned to be securely received between the first collar and the second collar such that when the pincher is in the closed position, the first and second collars prevent relative of the outer elongated shaft and the pincher along the longitudinal axis.

3. The minimally invasive surgical instrument of claim 2, further comprising:
an end effector shaft translatable through the outer elongated shaft, and a cutting blade shaft translatable through the end effector shaft.

4. The minimally invasive surgical instrument of claim 3, further comprising:
an end effector that is releasably coupled to the end effector shaft, wherein the end effector is releasable from the end effector shaft while the end effector shaft remains secured to the housing.

5. The minimally invasive surgical instrument of claim 4, wherein:
the end effector shaft is configured to be coupled to an actuator within the housing, and wherein translation of the actuator actuates the end effector, wherein the end effector shaft includes a collar that includes a plurality of fingers and the actuator includes a plurality of recesses, the fingers and the recesses being configured to mate with one another.

6. The minimally invasive surgical instrument of claim 5, wherein:
a locking mechanism releasably secures a connection member of the end effector to the end effector shaft.

7. The minimally invasive surgical instrument of claim 6, wherein:
the locking mechanism comprises a latch and a hook.

8. The minimally invasive surgical instrument of claim 6, wherein:
the locking mechanism comprises a camming member.

9. The minimally invasive surgical instrument of claim 8, wherein:
when the connection member is rotated in a first direction relative to the end effector shaft, the camming member locks the connection member to the end effector shaft and when the connection member is rotated in a second direction relative to the end effector shaft, the camming member unlocks the connection member from the end effector shaft.

10. The minimally invasive surgical instrument of claim 6, wherein:
the locking mechanism locks the end effector to the end effector shaft when by being rotated in a first direction relative to the other and is unlocked by being rotated in a second direction.

11. The minimally invasive surgical instrument of claim 6, wherein:
the connection member is threadably secured to the end effector shaft,

12. The minimally invasive surgical instrument of claim 6, wherein:
the connection member is frictionally secured within the end effector shaft,

13. The minimally invasive surgical instrument of claim 6, wherein:
the connection member includes an L-shaped groove for receiving and releasably locking a protrusion extending from the end effector.

14. The minimally invasive surgical instrument of claim 6, wherein:
the end effector includes an electrically conductive surface configured to cauterize tissue coming into contact with the end effector.

15. The minimally invasive surgical instrument of claim 6, wherein:
the end effector shaft comprises at least two shafts that are telescopically arranged such that a length of the end effector shaft is adjustable.

16. The minimally invasive surgical instrument of claim 15, further comprising:
a locking mechanism to secure the at least two shafts in a relative position with respect to one another.

17. The minimally invasive surgical instrument of claim 16, wherein:
the at least two shafts include an outer shaft and an inner shaft, wherein the locking mechanism includes outwardly biased buttons that are provided on the inner shaft and receptacles that are provided on the outer shaft, the receptacles being configured to receive the outwardly biased buttons.

18. The minimally invasive surgical instrument of claim 16, wherein;
the at least two shafts include an outer shaft and an inner shaft, wherein the locking mechanism includes a track provided on the outer shaft and a protrusion provided on the inner shaft, the protrusion being configured to travel along the track when the two shafts are positioned in a first rotational position relative to one another such that the two shafts may slide relative to one another and are inhibited form sliding relative to one another when the two shafts are in a second rotational position relative to one another.

19. The minimally invasive surgical instrument of claim 16, wherein:
the locking mechanism includes a hook and a latch.
